(19) United States
(12) Patent Application Publication
(10) Pub. No.: US 2010/0129691 A1
(43) Pub. Date: May 27, 2010

(54) ENHANCED PRODUCT GAS AND POWER EVOLUTION FROM CARBONACEOUS MATERIALS VIA GASIFICATION

(75) Inventors: John Dooher, Garden City, NY (US); Marco Castaldi, Yonkers, NY (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: GOOD EARTH POWER CORPORATION, Lake Buena Vista, FL (US)

(21) Appl. No.: 12/624,465

(22) Filed: Nov. 24, 2009

Related U.S. Application Data
(60) Provisional application No. 61/117,988, filed on Nov. 26, 2008.
(60) Provisional application No. 61/255,143, filed on Oct. 27, 2009.

(51) Int. Cl. H01M 8/18; C01B 3/36; C07C 27/06; B01J 19/00; B01J 10/00; H01M 8/04

(52) U.S. Cl. 429/17; 252/373; 518/702; 422/198; 422/189; 429/20

(57) ABSTRACT

Provided are devices and related methods for enhanced gasification of carbonaceous material in which supplemental carbon dioxide is introduced to the gasifier. The gasified carbonaceous material can then be used as a syngas or further processed into hydrocarbon form. The hydrocarbon can then be used in a fuel cell to produce electrical power or used in a traditional combustion process.

### RELATED APPLICATIONS

The present application claims the benefit of U.S. Application Ser. No. 61/117,988, filed Nov. 26, 2008, and U.S. Application Ser. No. 61/255,143, filed Oct. 27, 2009, the entireties of which are incorporated by reference herein for all purposes.

### TECHNICAL FIELD

The present application relates to the field of gasification and the production of hydrocarbons and power from carbonaceous materials.

### BACKGROUND

The growth in global population and in energy-demanding technology has created an increasing demand for energy. While a large proportion of the world's energy is derived from hydrocarbon fuels that are laboriously extracted from various geologic formations, many in the Middle East, the increased demand for such fuels has driven up the price of such fuels and has even raised questions regarding whether the demand for such fuels may at some point outstrip the supply.

Ongoing questions regarding climate change and so-called greenhouse gases have also increased interest in the capture and sequestration of carbon dioxide, a frequent by-product of existing power generation systems. The confluence of these energy and environmental issues has generated a renewed motivation for developing new concepts for coal/biomass/waste to energy gasification. Accordingly, there is a need in the field for methods and devices capable of converting carbonaceous material, such as waste, to energy.

### SUMMARY

Applicants have developed systems and methods for enhanced product gas evolution from indirect and/or partial combustion of carbonaceous materials, such as, for example, coal, biomass, and/or waste. While not being bound to any single theory of operation, the purposeful introduction of carbon dioxide at the gasification stage of the disclosed methods and systems enables the user to tune the composition of the products evolved from the claimed systems and methods. For example, the introduction of carbon dioxide allows for production of compositions having a controllable range of carbon and hydrogen content. The increased ability to recover carbon from a carbonaceous feed material can, in some embodiments, result in more efficient power production, as more carbonaceous fuel can be recovered from a given amount of carbonaceous feed material.

In one illustrative embodiment, a system for producing a syngas from carbonaceous material comprises a gasifier, combustion chamber, and a controller. The gasifier may comprise at least one inlet and one outlet. The gasifier outlet is suitably in fluid communication with the combustion chamber. The combustion chamber emits a product stream comprising, inter alia, at least carbon dioxide. A conduit communicates at least a portion of the carbon dioxide emitted from the combustion chamber to the least one inlet of the gasifier. A controller controls and modulates the carbon dioxide entering the gasifier such that the carbon dioxide entering the gasifier represents between about 10 vol % and 50 vol % of all material entering the gasifier.

In an illustrative method, a feed stream comprising a carbonaceous material is introduced into a gasifier. The gasifier at least partially gasifies at least a portion of the carbonaceous material to give rise to a syngas product stream comprising at least carbon monoxide. The syngas product stream is further processed to give rise to a second product stream comprising at least carbon dioxide. In an exemplary method, the syngas product stream may be further processed by, for example, a combustion chamber. At least a portion of the carbon dioxide of the second product stream is communicated to the gasifier such that carbon dioxide constitutes between about 10 vol % and about 50 vol % of all material introduced to the gasifier.

According to an illustrative embodiment, a method for producing hydrocarbon from carbonaceous material comprises introducing a volume of carbonaceous material into a gasifier. At least a portion of the material is gasified so as to evolve a first product stream comprising at least carbon monoxide. Carbon monoxide of the product stream is processed with hydrogen, water, or both to give rise to a second product stream. In some embodiments, the second product stream comprises hydrogen, water, carbon dioxide, a hydrocarbon, or some combination thereof. Carbon dioxide from the second product stream is fed into the gasifier such that carbon dioxide constitutes between about 10 vol % and about 50 vol % of all material introduced to the gasifier.

In exemplary embodiments of the disclosed systems and methods, syngas produced from an indirect or partial combustion gasifier may be used as a source of carbon dioxide which may be generated by combustion in a combustion chamber. The carbon dioxide may be used to provide heat either for gasification or for power generation. The carbon dioxide may also be used as a reactant for the gasification process.

In exemplary embodiments of the disclosed systems and methods, injection of carbon dioxide into the gasifier in varying amounts (e.g., from 20-30% by volume of the material introduced into the gasifier) results in a variation of the amounts of generated carbon monoxide, hydrogen, and methane via the interplay of, inter alia, the Boudouard reaction (C+CO₂=2CO), the water gas shift reaction (CO+H₂O=CO₂+H₂), and the methanation or first Fischer-Tropsch reaction (CO+3H₂=CH₄+H₂O).

Injection of carbon dioxide into the gasifier in varying amounts (e.g., 20-30% by volume of the material introduced into the gasifier) enhances access of the reactants, steam and carbon dioxide, to the feedstock. Without being bound to any single theory of operation, the gasification of coal, biomass, or other materials is accelerated by way of the increased porosity effected in the carbonaceous feed material brought about by injected or recycled carbon dioxide. The carbon dioxide effects enhanced conversion of a porous carbonaceous material; as the carbonaceous pores react, more surface area is exposed for reaction and the reaction proceeds more quickly. In addition, the presence of carbon dioxide and its reaction with the carbon in the feedstock can effect the equilibrium of the exothermic water gas shift reactions which can provide energy to accelerate the gasification reactions.

Injection of carbon dioxide into a partial combustion gasifier reduces oxygen requirements for specific syngas compositions compared to no carbon dioxide injection since oxygen can be supplied by the carbon dioxide.

In an exemplary embodiment, by use of an indirectly heated gasifier, either entrained flow of fluidized beds, for example, air can be used to oxidize the fuel used to heat the gasifier to drive the endothermic gasification reactions, yet produce a high quality syngas without nitrogen dilution. The syngas can then be oxidized in an oxy-combustor or in a fuel cell—such as a solid oxide fuel cell—to produce power and thereby produce a pure carbon dioxide stream which can then be recycled as a gasifier reactant.

The carbon dioxide can be fed to the gasifier either as a separate gas stream, or condensed under pressure to form a coal/liquid carbon dioxide slurry. A biomass or waste to energy/water or carbon dioxide slurry may also be fed into a high pressure gasifier; the envisioned embodiments are applicable to a range of feedstocks and are not limited to solids or liquids.

In one illustrative embodiment, provided are methods of producing a syngas from a carbonaceous material, the methods including introducing into a gasifier a feed stream comprising a carbonaceous material; at least partially gasifying at least a portion of the carbonaceous material to give rise to a syngas product stream comprising at least carbon monoxide; further processing the syngas product stream to give rise to a second product stream comprising at least carbon dioxide; and conveying at least a portion of the carbon dioxide of the second product stream to the gasifier such that carbon dioxide constitutes between about 10 vol % and about 50 vol % of all the material introduced to the gasifier.

In another illustrative embodiment, provided are systems for producing a hydrocarbon from a carbonaceous material, comprising a gasifier having at least one inlet and one outlet, the outlet of the gasifier being in fluid communication with a combustion chamber, the combustion chamber emitting a product stream comprising at least carbon dioxide; a conduit placing at least a portion of the carbon dioxide emitted from the combustion chamber in fluid communication with the least one inlet of the gasifier, a controller capable of modulating the carbon dioxide entering the gasifier such that the carbon dioxide entering the gasifier represents between about 10 vol % and 50 vol % of all material entering the gasifier.

Further provided are methods of producing a hydrocarbon from a carbonaceous material, comprising introducing a volume of carbonaceous material into a gasifier; at least partially gasifying at least a portion of that material so as to evolve a first product stream, the first product stream comprising at least carbon monoxide; processing at least a portion of the carbon monoxide of the product stream with hydrogen to give rise to a second product stream comprising at least carbon dioxide and a hydrocarbon; and introducing carbon dioxide into the gasifier such that carbon dioxide constitutes between about 10 vol % and about 50 vol % of all material introduced to the gasifier.

The increased ability to recover carbon from a carbonaceous feed material can, in some embodiments, result in more efficient power production, as more useful carbonaceous fuel can be recovered from a given amount of carbonaceous feed material. Applicants disclose processing carbon in a gasifier to generate syngas, communicating the syngas to a fuel cell, and communicating carbon dioxide generated by the fuel cell back to the gasifier where it is used in the gasification process. Thus, Applicants disclose generating electric power from syngas, and recycling carbon dioxide from the electric power production to a gasification process in order to enhance the syngas production processes.

In a disclosed illustrative system, an output of a gasifier is fluidly coupled to a fuel cell input. An output of the fuel cell is fluidly coupled to an input of the gasifer. In an illustrative process, within the gasifier carbonaceous material is gasified in the presence of carbon dioxide so as to give rise to a fuel product comprising carbon monoxide and hydrogen. Carbon monoxide and the hydrogen from the gasifier is fluidly communicated to the fuel cell, which converts at least a portion of the fuel product to at least electrical energy and a second product stream comprising carbon dioxide and water. At least a portion of the carbon dioxide evolved from the fuel cell is fluidly communicated to the input of the gasifier.

Virtually any fuel cell adapted to operate as described herein may be used in the disclosed systems and methods. For example, a fuel cell that is adapted to receive and process carbon monoxide in order to generate electricity and carbon dioxide may be suitable for use in an exemplary embodiment.

Any gasifier adapted to operate as described herein may be used in the disclosed systems and methods. For example, a gasifier system such as is disclosed in U.S. application No. 61/117,988, filed Nov. 26, 2008, incorporated herein by reference in its entirety, may be used in the systems and methods disclosed herein. As disclosed in detail in that application, gasifiers can be used as part of a system that produces hydrocarbons from carbonaceous material wherein the production can be enhanced by controlled introduction of carbon dioxide into the gasifier.

Injection of carbon dioxide into the gasifier in varying amounts (e.g., from 20-30% by volume of the material introduced into the gasifier) results in a variation of the amounts of generated carbon monoxide, hydrogen, and methane via the interplay of, inter alia, the Boudouard reaction (C+CO₂=2CO), the water gas shift reaction (CO+H₂O=CO₂+H₂), steam gasification (H₂O+C=H₂+CO), and the methanation or first Fischer-Tropsch reaction (CO+3H₂=CH₄+H₂O). Applicants have noted that controlled injection of carbon dioxide into the gasifier in varying amounts (e.g., 20-30% by volume of the material introduced into the gasifier) may enhance access of the reactants, steam and carbon dioxide, to the feedstock.

Without being bound to any single theory of operation, the gasification of coal, biomass, or other materials is accelerated by way of the increased porosity effected in the carbonaceous feed material brought about by injected or recycled carbon dioxide. The carbon dioxide effects enhanced conversion of a carbonaceous material that is suitably porous; as the carbonaceous pores react, more surface area is exposed for reaction and the reaction proceeds more quickly. In addition, the presence of carbon dioxide and its reaction with the carbon in the feedstock can effect the equilibrium of the exothermic water gas shift reactions which can provide energy to accelerate the gasification reactions. Moreover, injection of carbon dioxide into a partial combustion gasifier reduces oxygen requirements for specific syngas compositions compared to no carbon dioxide injection since oxygen can be supplied by the carbon dioxide.

This Summary is provided to introduce a selection of concepts in a simplified form that are further described below in the Detailed Description of Illustrative Embodiments. This Summary is not intended to identify key features or essential features of the claimed subject matter, nor is it intended to be used to limit the scope of the claimed subject matter. Other features are described below.

### BRIEF DESCRIPTION OF THE DRAWINGS

The Summary, as well as the following detailed description, is further understood when read in conjunction with the appended drawings. For the purpose of illustrating the potential embodiments, there is shown in the drawings exemplary embodiments; however, the potential embodiments are not limited to the specific methods, compositions, and devices disclosed. In addition, the drawings are not necessarily drawn to scale. In the drawings:

FIG. 1 depicts a sample, non-limiting arrangement of process modules and reactant flow.

FIG. 2 depicts a flow chart of an exemplary process for gasification processing.

FIG. 3 depicts a sample, non-limiting arrangement of process modules and reactant flow;

FIG. 4 illustrates the effect supplemental CO₂ has on carbon conversion of steam gasification of bituminous coal; and

FIG. 5 illustrates the effect supplemental CO₂ has on carbon conversion of steam gasification of low-rank coals.

### DETAILED DESCRIPTION OF ILLUSTRATIVE EMBODIMENTS

Applicants have developed systems and methods for enhanced product gas evolution from indirect and/or partial combustion of carbonaceous materials.

In an illustrative embodiment, a stream of carbonaceous material is introduced into a gasifier. The gasifier processes at least a portion of the carbonaceous material and generates a syngas product stream comprising carbon monoxide. The syngas product stream is further processed to generate a second product stream comprising at least carbon dioxide. In an exemplary method, the syngas product stream may be processed by, for example, a combustion chamber. In an embodiment, the carbon monoxide is processed with water to form carbon dioxide. The carbon dioxide is fed to the gasifier such that carbon dioxide suitably constitutes between about 10 vol % and about 50 vol % of all material introduced to the gasifier.

By reusing carbon dioxide in the gasification step, the exemplary systems and methods may be used to minimize carbon dioxide emissions from the gasifier. Furthermore, and as described below, by controlling the amount of carbon dioxide that is introduced into the stream, the disclosed embodiments are capable of producing a variety of syngas compositions suitable for a variety of applications, such as transportation, small scale energy production, and distributed power. Still further, the disclosed systems and methods provide for minimizing particular output components such as methane or other contaminants.

### Exemplary Processing Arrangement

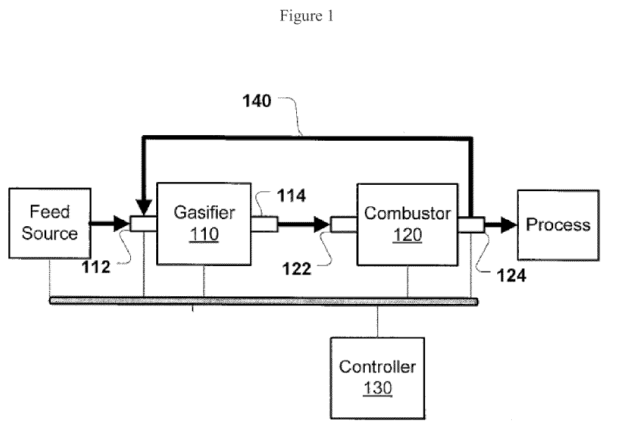

FIG. 1 depicts a sample, non-limiting arrangement of process modules and reactant flow. As illustrated in FIG. 1, an exemplary processing system comprises gasifier 110 communicatively coupled to combustor 120. Controller 130 communicatively coupled to combustor 120 and gasifier 110 and is adapted to control flow of material between the devices.

Gasifier 110 comprises intake 112, adapted to receive product flow into gasifier 110, and output 114, which is adapted to route flow out of gasifier 110 toward combustor 120. Reactants (feed) from a feed source are introduced to gasifier 110 at intake 112.

In gasifier 110, the feed may undergo one or more processes, including pyrolysis (in which volatiles are released and char is produced), combustion (volatiles and char reacting with oxygen to form carbon dioxide), gasification (char reacts with carbon dioxide and steam to produce carbon monoxide and hydrogen), and the reversible gas phase water gas shift reaction. Gasifier 110 may be any device suitable for providing the desired processing and may be, for example, a counter-current fixed bed, a co-current fixed bed, a fluidized bed, or an entrained flow gasifier. Other gasifier configurations will be known to those of ordinary skill in the art.

As illustrated in FIG. 1, the effluent from gasifier 110 is then fed to combustor 120. Combustor 120 comprises intake 122 for receiving product input and output 124 for outputting the results of processing. Combustor 120 is adapted to produce heat so as to combust or burn the materials received therein and may be any device suitable for the desired application.

Feedback conduit 140 communicates flow between combustor 120 and gasifier 110. In an embodiment, conduit 140 is in fluid communication with output 124 of combustor 120, as well as with intake 112 of gasifier 110. Thus, flow may be routed from output 124 of combustor 120 to intake 112 of gasifier 110. Other arrangements for conduit 140 are envisioned. For example, conduit 140 may communicate between output 122 into a separate receptacle of gasifier 110 other than intake 112.

Controller 130 may be a logic device, such as a programmable computing device, that is adapted to control the operation of gasifier 110, combustor 120, and conduit 140. In particular, controller 130 may be adapted to control the flow of output from combustor 120 to gasifier 110. Controller 130 may be adapted to control the operation of valves or other mechanisms so as to control the flows between devices. In an exemplary embodiment, controller 130 may comprise, for example, a computing processor, memory, and instructions stored in memory and executable by the processor to control operation of gasifier 110, combustor 120, and conduit 140 as described herein.

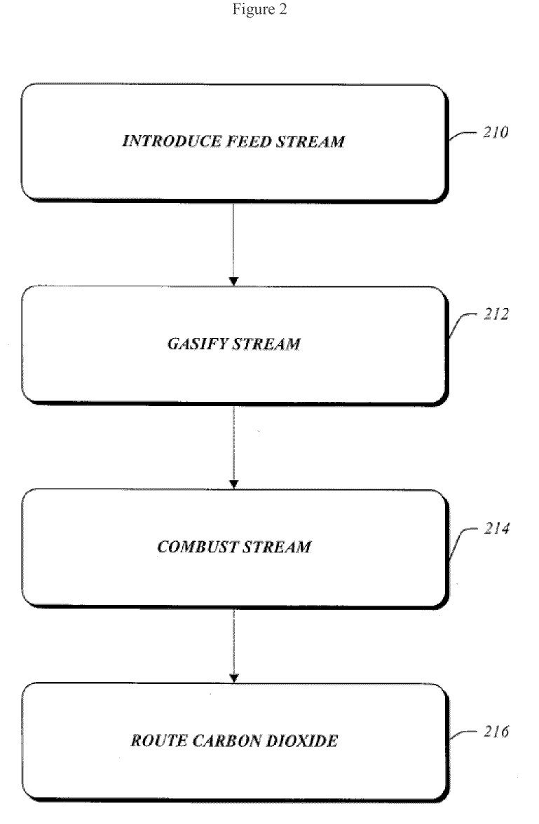

FIG. 2 provides a flow chart of a process for enhanced product gas evolution from indirect and/or partial combustion of carbonaceous materials. As shown in FIG. 2, at step 210, a feed stream is introduced into gasifier 110 via intake 112. The feed stream may be any carbonaceous material such as, for example, coal, biomass, waste, or any other suitable material.

At step 212, within gasifier 110, the feed stream undergoes gasification processing. Gasification processing may comprise, for example, reacting the feed stream at high temperatures with other reactants. The result stream of the gasification process is evacuated through output port 114. The result stream may comprise a syngas product stream which, in an exemplary embodiment comprises at least carbon monoxide.

At step 214, the syngas product stream is received into combustor 120 and undergoes combustor processing. The processing results in a second product stream that comprises at least carbon dioxide. The second product stream exits combustor 120 at output 124.

At step 216, controller 130 controls conduit 140 so as to route at least a portion of the carbon dioxide from combustor back to gasifier 110. In an embodiment, the carbon dioxide may be recovered or purified before being conveyed to gasifier 110. In an exemplary embodiment, controller 130 operates to control the amount of carbon dioxide flow from combustor 120 to gasifier 110 such that the carbon dioxide constitutes between about 10 vol % and about 50 vol % of all material introduced to gasifier 110. Any portion of carbon dioxide from combustor that is not recycled to gasifier 110 may be sequestered, stored, or otherwise disposed of.

### Additional Description

There are numerous potential embodiments for the above described principles. For example, in an exemplary embodiment, a method of producing a syngas from a carbonaceous material includes introducing into a gasifier a feed stream comprising a carbonaceous material, at least partially gasifying at least a portion of the carbonaceous material to give rise to a syngas product stream comprising at least carbon monoxide, further processing the syngas product stream to give rise to a second product stream comprising at least carbon dioxide; and conveying at least a portion of the carbon dioxide of the second product stream to the gasifier such that carbon dioxide constitutes between about 10 vol % and about 50 vol % of all the material introduced to the gasifier.

A variety of carbonaceous materials are considered suitable for introduction to the gasifier; organic materials, such as biomass, are considered particularly suitable. Other carbonaceous materials, including coal, polymers, plastic, rubber, and the like are all suitable. In some embodiments, the feed to the gasifier may include sewage sludge, municipal solid waste, agricultural waste, and the like, including chicken litter, pig manure and cow dung.

In some embodiments, the feed to the gasifier is thermally pretreated, chemically pretreated, or both, to render the feed more suitable for gasification.

Gasification is suitably performed in the range of from about 800 deg. C. to about 1600 deg. C. Lower gasification temperatures may be employed in certain types of gasifiers such as indirectly heated fluidized bed systems; higher temperatures are often seen in partial oxidation entrained flow gasifiers. Entrained gasifiers can suitably operate at least partially by indirect heating. In order to perform the Boudouard reaction, CO2+C=2CO, temperatures of 800 deg. C. or greater are typically employed. Typical gasification processes are performed at about 800 deg. C. or greater.

Gasification is performed at a wide range of pressures, from e.g., about 1 atm to about 72 atm. The optimal process pressure will be apparent to those ordinary skill in the art and will depend on the desired composition of the product stream from the gasifier and desired throughput. Gasification may be carried out under reduced pressure, i.e., pressure of less than 1 atm.

In some embodiments, water is added to the gasifier during the gasification process. Steam may also be added to the gasifier, suitably such that the molar ratio of steam to carbon present in the material introduced to the gasifier is in the range of from about 1:10 to about 5:1, or even 1:1. A ratio of 1:10 is useful, for example, in a partial oxidation gasifier where a product gas comprising CO is desired; a ratio of 1:1 may be useful where coal/slurry is being gasified.

In other embodiments, partial oxidation with little to no water or steam is performed to produce CO and hydrogen, which products evolve from dissociation of the hydrocarbons in the feedstock. While some forms of plasma gasification achieve this, it is nevertheless expected that introducing carbon dioxide into the reaction will enhance the resulting product gas evolution.

A variety of gasification methods are considered suitable. Gasification may be accomplished by partial oxidation, by steam gasification, by gasification where solar energy provides indirect heating of reactants, feed, and process modules, by plasma-assisted gasification where added CO2 assists in the gasification. Combinations of gasification methods are also considered suitable.

Various mechanical and hydrodynamic-based configurations of gasifiers are considered suitable. Downdraft and updraft gasifiers are considered suitable, as are fixed bed, moving bed, fluidized bed, bubbling bed, circulating fluidized bed, fast fluidized bed (including a transport reactor having enhanced circulation), entrained flow (both slurry and dry feed), and gasifiers involving partial oxidation or indirect heating are all considered suitable.

In some embodiments, wherein conveying at least a portion of the second product stream includes separating carbon dioxide from the second product stream and transporting that carbon dioxide to the gasifier, although in some embodiments, a portion of the product stream can be recycled back to the gasifier. In some embodiments, at least a portion of the carbon dioxide of the second product stream is sequestered. Separation and sequestration may be accomplished by, inter alia, chemical absorption or separation (e.g., monoethanolamine), membrane separation, and other techniques known to those of ordinary skill in the art.

The carbon dioxide conveyed to the gasifier (e.g., from the second product stream) can be in liquid form, to which liquid carbon dioxide can be added carbonaceous material, such as the feed material. The disclosed methods also, in some embodiments, include combining the carbon dioxide of the syngas product stream conveyed to the gasifier with at least a portion of the feed stream prior to introduction to the gasifier. In such embodiments, the liquid carbon dioxide is used to convey feed material into the gasifier, although such conveyance can also be accomplished with gaseous carbon dioxide.

Gasification is suitably performed in the presence of oxygen. The oxygen present in the gasifier may be that oxygen that is present under ambient conditions. Depending on the user's needs, the gasification may also be performed with or without added oxygen.

As described elsewhere herein, the syngas product stream includes carbon dioxide. Suitably, at least a portion of the carbon dioxide of the syngas product stream is conveyed to the gasifier such that carbon dioxide constitutes between about 10 vol % and 50 vol %, or between about 15 and 45 vol %, or even between 20 and 30 vol % of all material introduced to the gasifier. Embodiments wherein carbon dioxide comprises between about 20 and 30 vol % of all material entering the gasifier are considered especially suitable.

Depending on the user's needs, at least a portion of the carbon dioxide of the second product stream may be conveyed to the gasifier such that carbon dioxide constitutes between about 20 vol % and 30 vol % of all material introduced to the gasifier. The balance between the carbon dioxide introduced to the gasifier from the first product stream, the syngas product stream, or both, will depend on the user's needs.

In some embodiments, material is introduced continuously to the gasifier. In other embodiments, material is introduced in a batch or semi-continuous process. Hydrogen, oxygen, or both may be introduced to or removed from the gasifier or other process modules, such as combustion chambers.

Heat evolved from further processing the syngas may, in some embodiments, be returned to the gasifier or otherwise used to heat the feed stream. Transferring heat from one process module to another thus reduces the disclosed methods' need for externally-supplied utilities.

The methods include further processing of the syngas product stream, which further processing, in some embodiments, includes subjecting the syngas product stream to a Fischer-Tropsch reaction scheme. As described elsewhere herein, this reaction scheme enables the production of hydrocarbons from carbon monoxide. In some embodiments, the further processing includes separating carbon dioxide from the syngas product stream, sequestering carbon dioxide from the syngas product stream, or both. The further processing, in certain embodiments, can include, for example, combustion.

A variety of products may be manufactured by the further processing of the syngas. Such products include ammonia, plastic, polymers, cellulosic polymers, solvents, resins, naphtha, wax, alcohols, ethylene, acetate, propylene, and the like. The further processing may, for example, involve the production of propane via the Fischer-Tropsch reaction, which propane is then converted to propylene through a catalytic conversion process.

Systems for producing a hydrocarbon from a carbonaceous material are also provided. The systems suitably include gasifiers having at least one inlet and one outlet, the outlet of the gasifier being in fluid communication with a combustion chamber. The combustion chamber suitably emits a product stream comprising at least carbon dioxide.

The systems also include at least one conduit placing at least a portion of the carbon dioxide emitted from the combustion chamber in fluid communication with the least one inlet of the gasifier and a controller capable of modulating the carbon dioxide entering the gasifier such that the carbon dioxide entering the gasifier represents between about 10 vol % and 50 vol % of all material entering the gasifier.

Suitable gasifiers are characterized as indirectly heated or a partial combustion gasifiers, which gasifiers are described in additional detail elsewhere herein. In some embodiments, the gasifier is a fuel cell, which fuel cell is suitably capable reforming input material into a gas. Solid oxide fuel cells are considered especially suitable, because the products of such fuel cells can be recycled for participation in other reactions and process steps.

Some embodiments of the systems include a device capable of continuously supplying supply of feed material to the gasifier, such as a conveyor, a pipe, and the like. The systems may also suitably include a device capable of separating carbon dioxide from the product stream emitted from the combustion chamber. Such devices include chemical absorbants, membranes, and the like.

The systems also, in some embodiments, include a conduit that places the combustion chamber in fluid communication with a vessel capable of containing or sequestering carbon dioxide. The vessel suitably includes one or more materials capable of immobilizing the carbon dioxide, although the vessel may also serve to contain the carbon dioxide. Such vessels may be underground, but may also be transportable tanks or similar containers. The systems may also include vessels capable of containing hydrocarbons evolved from the claimed methods, which vessels may be connection with the combustion chamber.

The systems may also include various other processing modules. For example, the system may include one or more reactor vessels—and associated catalysts—for further processing gasifier product streams. Such reactors may include, for example, a reactor capable of effecting a Fischer-Tropsch reaction on carbon monoxide, as well as reactors capable of effecting the water-gas shift reaction on carbon monoxide, water, carbon dioxide, and hydrogen. Methanation reactors—capable of converting carbon and water to methane and carbon dioxide—are also useful in the described systems, as are reactors capable of effecting steam reforming reactions and the Boudouard (C+CO₂=2CO) reactions. Reactors capable of other conversion reactions (e.g., alkane to alkene) reactions are also useful.

Also provided are methods of producing a hydrocarbon from a carbonaceous material. The methods suitably include introducing a volume of carbonaceous material into a gasifier, at least partially gasifying at least a portion of that material so as to evolve a first product stream, the first product stream comprising at least carbon monoxide; processing the carbon monoxide of the product stream with hydrogen to give rise to a second product stream comprising at least carbon dioxide and a hydrocarbon; and introducing carbon dioxide into the gasifier such that carbon dioxide constitutes between about 10 vol % and about 50 vol % of all material introduced to the gasifier.

As described elsewhere herein, the feed stream suitably includes an organic material, such as biomass. Municipal waste, agricultural waste, coal, and other materials described elsewhere herein are all considered suitable.

Gasification is performed in the range of from about 800 deg C. to about 1600 deg. C., or in the range of from about 900 to about 1400 deg C., or even in the range of from about 1000 to about 1100 deg C. The gasification may be performed at from about 1 atm to about 72 atm, as described elsewhere herein. Oxygen may, in some embodiments, be added to the gasifier, during later processing of the gasifier's product stream, or both.

Water, steam, or both may be added to the gasifier, and steam is suitably present in a molar ratio or steam to carbon present in the gasifier of from about 1:10 to about 5:1.

Gasification is suitably accomplished by partial oxidation. Other methods of accomplishing gasification are set forth elsewhere herein.

The methods, in some embodiments, include sequestering or otherwise storing at least a portion of the carbon dioxide of the second product stream. In some embodiments, essentially all of the evolved carbon dioxide is recycled to the gasifier; in others, little evolved carbon dioxide is recycled to the gasifier.

The carbon dioxide can be introduced to the gasifier in liquid form. As described elsewhere herein, feed material or other additives can be added to the liquid carbon dioxide for introduction into the gasifier.

Processing of a product stream to give rise to a hydrocarbon is suitably performed to give rise to a hydrocarbon having at least two carbon atoms, or, in some embodiments, to give rise to a hydrocarbon having at least six carbon atoms. The processing may be performed via a fluidized bed, although it may also be performed in a packed-bed reactor, a porous monolith, or other reactors known to those of skill in the art.

The first product stream, as described elsewhere herein, suitably includes carbon dioxide. Introducing the carbon dioxide into the gasifier is suitably performed such that carbon dioxide constitutes between about 20 vol % to about 30 vol % of all material introduced to the gasifier. The introduction of carbonaceous material to the gasifier is suitably performed continuously.

Introducing carbon dioxide into the gasifer, in some embodiments, includes conveying at least a portion of the carbon dioxide of the second product stream to the gasifier such that carbon dioxide constitutes between about 20 vol % to about 30 vol % of the volume of material introduced to the gasifier. Hydrogen, oxygen, or both may also be introduced to the gasifier or removed therefrom, depending on the user's needs and the desired composition of gasifier product. Heat evolved from processing the first product stream may be transferred to the gasifier, as described elsewhere herein. In some embodiments, the methods include integration of process modules with one another so as to minimize the methods' overall utility consumption.

### Exemplary Processing Embodiments

The following embodiments are exemplary of potential embodiments, but do not limit its scope of embodiments. It is understood by those of skill in the art that various features of the disclosed embodiments can be combined, rearranged, or otherwise modified to produce additional embodiments that are within the scope of the envisioned embodiments.

### EXAMPLE 1

The sample reaction scheme shown below illustrates one possible reaction scheme that may be employed consistent with the embodiments disclosed herein to produce a hydrocarbon.

The Fischer-Tropsch (FT) reaction,

(2n+1)H₂+nCO=CₙH₂ₙ₊₂+nH2O,

can be used to produce a hydrocarbon, CₙH₂ₙ₊₂, by (as shown) combusting hydrogen (H2) with carbon monoxide (CO). In an exemplary embodiment, the hydrocarbon may be a liquid at ambient conditions where n is at least 5 or 6.

A carbonaceous material can be gasified, with the application of steam, in order to generate hydrogen and carbon monoxide gas:

nC+nH₂O=nH₂+nCO

At the same time, the water gas shift equilibrium reaction may be employed to govern the partition between carbon monoxide, water, hydrogen, and carbon:

CO+H₂O=CO₂+H₂

The Boudouard reaction also describes the conversion of carbonaceous material to carbon monoxide:

C+CO₂=2CO

In an exemplary embodiment, these reactions may be coupled to produce a hydrocarbon.

Addition of steam

nC+nH2O=nH₂+nCO

The water gas shift reaction yields

(2n/3n+1)nCO+(n+⅓n+1)nH₂O+nH₂+(n+⅓n+1)nCO=(2n/3n+1)nCO+(4n+⅔n+1)nH₂+(n+⅓n+1)nCO₂=(Fischer-Tropsch)=(2n/3n+1)CₙH₂ₙ₊₂+(n+⅓n+1)nCO₂+(2n/3n+1)nH₂O

Addition of CO₂ provides the following:

(2n/3n+1)nC+(n+½)H₂+(n−½n)nCO₂=2nCO+(n+1)H₂O

The water-gas shift reaction then yields:

nH₂O+2nCO+(n+1)H₂O=(2n+1)H2+nCO+nCO2=(Fischer-Tropsch)=CₙH₂ₙ₊₂+nCO₂+nH₂O

From the above, it can be seen that introduction of additional carbon dioxide, as described above and as implemented in exemplary embodiments, can be used to bias the equilibrium of the various reactions shown above toward the desired product mix. For example, addition of carbon dioxide can be used to push the water-gas shift reaction toward production of carbon monoxide, which in turn is converted to a hydrocarbon via the Fischer-Tropsch reaction.

In the above scheme, careful modulation of the amount of carbon dioxide can thus be used to control hydrocarbon production. More generally, modulation of the carbon dioxide can also control the amount of hydrogen, methane, and carbon monoxide. Careful modulation of the various reactions is also necessary to optimize reaction yield.

### EXAMPLE 2

Gasification of biomass and other carbonaceous feed materials can in some instances result in the formation of char and tar, either of which can clog and damage processing equipment. Formation of char is also indicative of incomplete conversion of the carbonaceous feed materials.

The addition of carbon dioxide to a gasifier during biomass processing, as described herein, results in decreased char and tar formation. In particular, the disclosed methods for carbon dioxide recycling may result in enhance char conversion to a useful CO gas product. The decrease in char formation presents the benefit of reduced need for cleaning and maintenance of process equipment. The decrease in char formation also demonstrates a higher conversion of the carbonaceous starting material to carbon-containing gas that can then participate in one or more reactions to produce a hydrocarbon fuel.

The increased conversion effectively increases the amount of fuel that is ultimately produced from a given amount of carbonaceous starting material, thus giving rise to more efficient fuel production. Thus, introduction of carbon dioxide consistent with the systems and methods disclosed herein effectively increases the amount of useable energy that can be extracted from a given amount of carbonaceous material while reducing the formation of char and tar.

Furthermore, because the disclosed methods for recycling carbon dioxide provide a more complete use/conversion of the carbon, there is also a reduce need for steam, which has traditionally been used in such reactions. The reduced need for steam results in using less water and less energy to raise steam. Thus, in the disclosed embodiments, carbon dioxide may be purposefully added to offset the need for steam and energy. By constrast, some existing processing methods seek to minimize the participation of carbon dioxide in the gasification process.

### EXAMPLE 3

Recycling carbon dioxide into a gasifier as described herein may be employed to effect the concentration of carbon monoxide evolved from gasifying a biomass material. Increasing the amount of carbon dioxide present in the gasifier results—as shown by the reaction schemes described above—in an increasing concentration of carbon monoxide in the product stream from the gasifier. The increased concentration of carbon monoxide resulting from recycling carbon dioxide as described herein increases the amount of carbon monoxide available to participate in subsequent, hydrocarbon-fuel forming reactions.

### EXAMPLE 4

Recycling carbon dioxide into a gasifier as described herein may be employed to effect the concentration of hydrogen evolved from gasifying a biomass material. Increasing the amount of carbon dioxide present in the gasifier results in a decreased concentration of hydrogen in the product stream from the gasifier. This decreased level of hydrogen in turn favors an increased level of carbon monoxide. The increased amount of carbon monoxide is then available to participate in subsequent, hydrocarbon-fuel forming reactions. Thus, the disclosed methods provide for the utilization of carbon dioxide to reduce hydrogen, which attributes enable tuning of the hydrogen to carbon monoxide ratio for optimal production of fuels via the Fisher-Tropsch reaction or other processes.

### EXAMPLE 5

Recycling carbon dioxide into a gasifier as described herein may also be employed to effect the concentration of methane evolved from gasifying a biomass material. For some applications, methane may be considered undesirable. For example, methane mixed with a carbon dioxide-containing product stream may create difficulties in sequestering the carbon dioxide because the sequestration system must be capable of retaining both carbon dioxide and methane. Increasing the amount of carbon dioxide present in the gasifier results in a decreased concentration of methane.

### EXAMPLE 6

Still further, recycling carbon dioxide into a gasifier as described herein may be employed to effect the rate at which a carbonaceous material is gasified with and without the introduction (via recycle) of carbon dioxide evolved from a subsequent reaction. Increasing the relative amount of carbon dioxide present in the gas produced by the gasifier thus increases the amount of carbon in the product stream that is then available for subsequent processing into fuel and other carbon-containing compositions.

Thus, as is described in the foregoing examples, the introduction of carbon dioxide consistent with the disclosed embodiments may be employed to effect the generation of various materials during carbon biomass processing. For example, introduction of carbon dioxide consistent with the disclosed embodiments offers advantages in gasification-based processes for producing hydrocarbon fuel from carbonaceous biomass materials.

The disclosed embodiments for recycling carbon dioxide in product gas evolution offers the prospect of improving energy utilization. For example, a wide range of product gas compositions can be generated in a controlled fashion from materials including coal, biomass, and sewage and agricultural wastes, by adjusting the reactant composition among water, carbon dioxide, and feedstock. Gaseous compositions suitable for production of liquid fuels are easily made employing the disclosed systems and methods.

The disclosed embodiments also provide for indirect heating of coal/biomass/waste, reactants, and associated process modules, which indirect heating can improve system control and efficiency. Furthermore, the disclosed embodiments provide the opportunity to operate with inexpensive air without product gas dilution, because carbon dioxide can be obtained from any convenient sequestration sources, such as, for example, underground tanks, absorbers, or other systems known to those in the art. Thus, the reduced oxygen requirements of the indirect gasifier can also lead to reduced operating costs.

The disclosed embodiments also offer the possibility of improved plant efficiencies. As compared to standard gasifier processing, because the disclosed methods offer reduced oxygen requirements and accelerated conversion reaction rates, pressurization is more easily accomplished, and the size of the system may be reduced for a given output. Without being bound to any particular value, it is expected that plant efficiencies greater than about 60% may be achieved. Furthermore, because of the enhanced reaction rates and the capability of being used with air blown gasifiers, the disclosed methods facilitate the application of economical small scale gasification applications.

Furthermore, the ability to tailor the product gases in terms of H₂/CO₂/CO/CH₄ ratios also make the disclosed methods ideally suited for a synthetic fuel industry where precise control over products is required. The disclosed systems and methods may be applied to large power generating systems where sequestration streams of carbon dioxide may already be readily available.

Because it departs from conventional gasifier systems, the disclosed embodiments are further unique in their ability to tune the composition of gasifier-evolved syngas and their value-added use of carbon dioxide holds the potential to change dramatically the coal/biomass/waste-to-energy field, chemical production, and the production of liquid fuels.

Thus, Applicants have disclosed systems and methods for enhanced product gas evolution from indirect and/or partial combustion of carbonaceous materials. The disclosed systems and methods offer the possibility of numerous advantages including, for example, energy savings, specific tailoring of the H₂ to CO ratio and CH₄ content for syngas production for multiple applications, more complete usage of the biomass as well as a demand for CO₂ that would otherwise be “landfilled.”

The disclosed gasifiers and related methods can also be adapted to operate with fuel cells. This in turn gives rise to systems and methods that generate electrical power from processing of carbonaceous materials, and cycle carbon dioxide that is created during the generation of electrical power to the processing of carbonaceous materials.

One example of such systems may comprise, for example, a gasifier which is fluidly coupled to a fuel cell. Within the gasifier, at least a portion of a carbonaceous material is at least partially gasified so as to give rise to a fuel product, which may comprise, for example, carbon monoxide and hydrogen.

The fuel product is communicated to a fuel cell where at least a portion of the fuel product is employed to generate at least electrical energy and a second product stream comprising carbon dioxide and water. At least a portion of the carbon dioxide evolved from the fuel cell is communicated back to the gasifier where it is used in the processing of carbonaceous material.

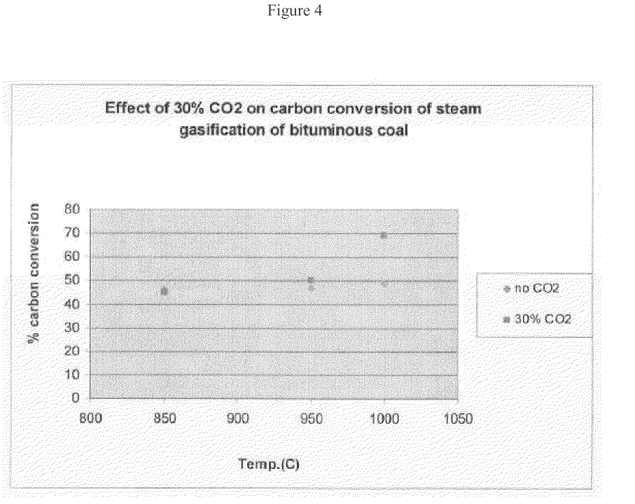

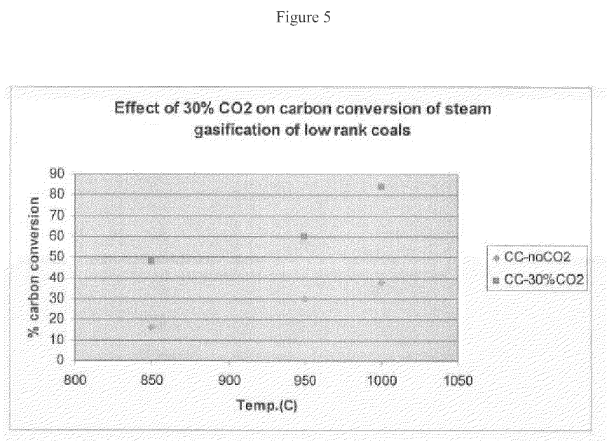

All or a portion of the product syngas generated at the gasifier is suitably combusted in a fuel cell, which may be any suitable fuel cell including, for example, a solid oxide fuel cell (SOFC). Molten carbonate and protonic ceramic fuel cells are also suitable. In the fuel cell, oxygen depleted air is produced at the cathode and combustion products (CP), which may include carbon dioxide and water vapor, are produced at the anode at a stream separate from the cathode air stream. Syngas that is not processed by the fuel cell may, for example, be further processed by the gasifier into hydrocarbon fuels, as described elsewhere herein. At least a portion of the combustion products, which may have a high temperature , e.g., 1000 deg. C., may be recycled to the gasification reactor to enhance the gasification process. For example, the gasifier may be operated as the indirectly heated gasifier that is described in U.S. Application No. 61/117,988, filed Nov. 26, 2008, incorporated herein by reference in its entirety, whereby adding CO₂ to the gasifier modifies the operation so that the system becomes a tunable catalytic gasifier (TCG). The performance and output of the gasifier is modulated by altering the amount of CO₂ that is introduced to the gasifier, as shown in attached FIG. 4 and FIG. 5.

In one non-limiting scenario, a feedstock of about 60 weight % dry coal in a water slurry is processed in the gasifier. In one mode of operation, approximately all (appx. 100%) of the syngas generated by the gasifier is communicated to the fuel cell and approximately twenty percent (20%) by volume of the combustion products generated by the fuel cell are recycled to the gasifier. In an alternative mode of operation, approximately 20% by volume of the syngas generated by the gasifier may be communicated to the fuel cell and approximately 100% of the combustion product from the fuel cell recycled to the gasifier. Either mode of operation results in about 25 volume % of carbon dioxide in the gasifier reactor, thereby enhancing the gasification product as described above and providing for complete gasification of the coal. Where a tunable catalytic gasifier such as is disclosed elsewhere herein is employed, approximately 40% by volume of the product syngas is catalytically combusted to drive the gasification reactions, and about 20% of the syngas can be recycled through the SOFC.

In another exemplary scenario, a dry coal feed (i.e., without water slurry) is processed in the gasifier. In a first mode of operating the system, all (100%) of the syngas generated by the gasifier is communicated to the fuel cell and about 50% by volume of the combustion products generated by the fuel cell is recycled back to the gasifier. In another mode of operation, about 50% of the syngas generated by the gasifier is communicated to the fuel cell and about 100% of the combustion products generated by the fuel cell is recycled to the gasifier. Either of these modes of operating the system may provide for a range of carbon dioxide of about 50 to about 75 vol % and of about 25 to about 50% water vapor in the gasifier reactor. Where a tunable catalytic gasifier such as is disclosed elsewhere herein is employed, about 40% by volume of the syngas is used for the heat for gasification, and 50% of the syngas can be recycled through the SOFC to provide tunability.

In another exemplary scenario, carbon dioxide gasification of coal may be employed. In a first mode of operating the system, approximately 100% of the syngas generated by the gasifier is communicated to the fuel cell and about 50% by volume of the combustion products generated by the fuel cell is recycled back to the gasifier. In another mode of operating the system, approximately 50% by volume of the syngas generated by the gasifier is communicated to the fuel cell and about 100% of the combustion products generated by the fuel cell is recycled back to the gasifier. Where a tunable catalytic gasifier is used, about 40% by volume of the syngas is used for the heat for gasification, and about 50% by volume can be recycled through the fuel cell to provide tunability.

Applicants have noted that controlled injection of carbon dioxide into the gasifier in varying amounts (e.g., 20-30% by volume of the material introduced into the gasifier) may enhance access of the reactants, steam and carbon dioxide, to the feedstock. The carbon dioxide effects enhanced conversion of a porous carbonaceous material; as the carbonaceous pores react, more surface area is exposed for reaction and the reaction proceeds more quickly. In addition, the presence of carbon dioxide and its reaction with the carbon in the feedstock can effect the equilibrium of the exothermic water gas shift reactions which can provide energy to accelerate the gasification reactions.

FIGS. 4 and 5 provide charts illustrating the enhanced reaction resulting from introduction of carbon dioxide into the gasification stage. FIG. 4 depicts a chart illustrating the effect of 30% by volume carbon dioxide on carbon conversion of steam gasification of bituminous coal. FIG. 5 depicts a chart illustrating the effect of 30% carbon dioxide on carbon conversion of steam gasification of low rank coals. In both charts, the percentage carbon conversion without the introduction of carbon dioxide is noted by the data points depicted with diamonds, while the percentage carbon conversion with the introduction of carbon dioxide is note by the data points depicted with squares. The effect of carbon dioxide into the feedstream is quite dramatic at 1000 C. This addition of CO₂ results in a surprisingly high carbon conversion at a relatively low temperature (about 1000 deg. C.) and short residence time (about 6 sec). In some embodiments, carbon dioxide represents less than about 30 vol % of the input to the gasifier. In others, carbon dioxide represents more than about 40 vol %, about 50 vol %, about 70 vol %, about 80 vol %, or even more than about 90 vol % of the input to the gasifier

According to another aspect of the disclosed embodiments, the enthalpy of the high temperature combustion products can supplement the heat the gasifier requires. In other words, the heat from the combusted products may be transferred to the gasifier and may assist in the gasification process. Additionally, the enthalpy from the combusted products created by the fuel cell may also be used to generate heat for use outside the system. For example, the enthalpy from the combusted products may be used in a combined heat and power (CHP) configuration, in which the system provides both heat (from the CP products) and power (from the fuel cell).

Extracting heat from the comparatively high temperature combustion products can also be used to reduce the temperature variations in the gasification process. Reducing such temperature variations extends the lifetime of the various materials used in the gasification process. Further, using the heat from the combusted process can also make the temperature within the gasifier (or other process units) more uniform, which can increase the lifetime of those process units. Fuel cell products can attain temperatures of hundreds of degrees Celsius.

In some embodiments, the fuel cell may include ceramics and other materials that tolerate high temperatures. The fuel cell may be integrated with the gasifier to simplify the overall system, and to facilitate heat transfer between the two units. For example, the fuel cell and gasifier may be constructed such that they are in physical contact with one another, or are located within the same enclosure.

The disclosed methods may be capable of operation with biomass, coal, and biomass/coal feedstocks, and supplementary landfill gas for additional heat and/or reactants such as carbon dioxide and methane. Supplemental landfill gas or other waste gases can be used as a source of heat by combustion to drive the endothermic gasification reactions.

The landfill gas (or other waste gas) can also be a source of carbon dioxide, with carbon dioxide being evolved either through combustion of the gas, steam reforming of the gas, or processing the gas with a solid oxide fuel cell or other fuel cell. The combustor, steam reformer, or fuel cell is then suitably in fluid communication with the gasifier to enable introduction of at least a portion of the carbon dioxide evolved from processing of the landfill gas to be introduced to the gasifier. Tar sands, sewage sludge, municipal solid waste, waste paper, and other carbon-containing waste materials capable of being processed for feeding to a gasifier are also suitable. Further, as described, the methods are amenable to use in combined heat and power (CHP) applications and also for distributed power generation, in which power is generated at numerous installations instead of large, consolidated generation facilities.

### Exemplary Embodiment

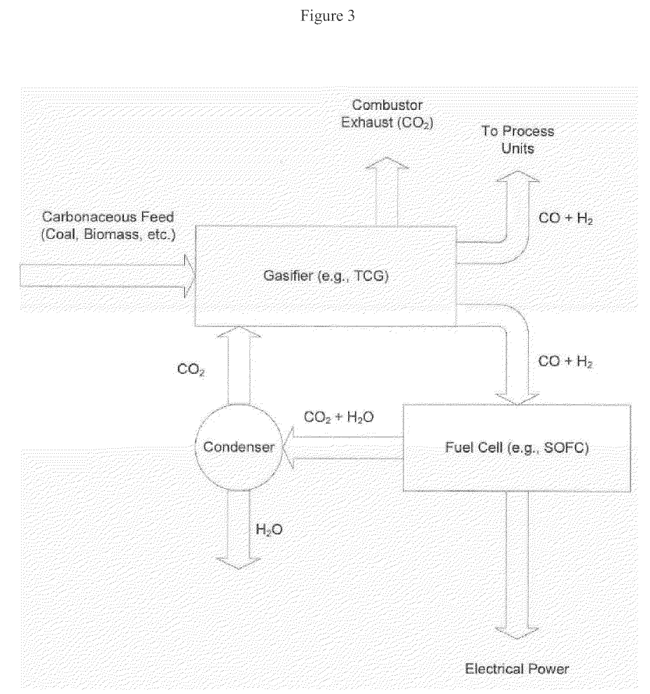

FIG. 3 depicts an exemplary embodiment of a system adapted to process carbonaceous material and generate electric power. In the illustrative embodiment shown in FIG. 3, a stream of carbonaceous material is introduced into a gasifier. The carbonaceous feed may be in a solid or slurry form. Air, carbon monoxide, oxygen, water, steam, or some combination of these is suitably fed to the gasifier.

The gasifier may be any device suitable for providing the desired processing and may be, for example, a counter-current fixed bed, a co-current fixed bed, a fluidized bed, an entrained flow gasifier, or any other suitable device. The gasifier may be a partial oxidation gasifier or an indirectly heated gasifier. The gasifier suitably includes an intake adapted to receive product flow into the gasifier, and an outlet, which is adapted to route flow out of the gasifier to the fuel cell. In a potential embodiment, the gasifier is embodied in the fuel cell, which fuel cell is suitably capable of reforming input material into a gas.

In the gasifier, the feed may undergo one or more processes, including pyrolysis (in which volatiles are released and char is produced), combustion (volatiles and char reacting with oxygen to form carbon dioxide), gasification (char reacts with carbon dioxide and steam to produce carbon monoxide and hydrogen), and the reversible gas phase water gas shift reaction. The gasifier processes at least a portion of the carbonaceous material and generates a syngas product stream comprising, for example, carbon monoxide and hydrogen gas. The gasifier also produces combustion exhaust—which may include carbon dioxide.

As illustrated in FIG. 3, gasifier product is fed to a fuel cell. The fuel cell may be any fuel cell adapted to operate consistent with the description herein. In many embodiments, a fuel cell converts carbon monoxide and hydrogen to water and carbon dioxide. In an exemplary embodiment, the fuel cell is suitably a solid oxide fuel cell (SOFC), although other types of fuel cells (identified elsewhere herein) may be used. A controller or other device may be used to modulate the flow of the syngas from the gasifier into the fuel cell.

The fuel cell converts the CO and H₂ from the syngas into electricity, CO₂, and water. The electricity is collected and may be used for any desired purpose. As shown in FIG. 3, CO₂ and/or water are recycled back to the gasifier unit. In one exemplary embodiment, a condenser is used to separate the water from the CO₂ before the CO₂ is recycled back to the gasifier. The condenser is suitably in fluid communication with the fuel cell as well as the gasifier. A controller or similar device may be used to modulate and control the amount of CO₂ that is recycled into the gasifier. Similarly, the condenser may be controlled so as to selectively control what amount, if any, of the water is included in the carbon dioxide that is recycled into the gasifier.

The combustion products including the carbon dioxide and water of the fuel cell are high-temperature. At least a portion of this heat may be extracted using a heat exchanger or other device. This extracted heat may be delivered to the gasifier to enhance that device's performance. The extracted heat may also be delivered to other process units, or may be used to provide heat to the process facility or to residential or commercial properties in the vicinity of the gasifier system.

The amount of carbon dioxide derived from the fuel cell that is recirculated to the gasifier may vary. For example, in some embodiments, from about 1 vol % to about 100 vol % of the carbon dioxide evolved from the fuel cell is recirculated to the gasifier. In other embodiments, from about 10 vol % to about 80 vol % of the carbon dioxide evolved from the fuel cell is recirculated to the gasifier, or from about 25 vol % to about 75 vol % of the carbon dioxide evolved from the fuel cell is recirculated to the gasifier. Alternatively, carbon dioxide suitably comprises in the range of from about 20 mol % to about 70 mol % of the carbon molar content of material fed to the gasifier. At least a portion of the carbon dioxide present in the product stream from the fuel cell may be isolated by, for example, condensing at least a portion of the product stream so as to separate the carbon dioxide product from water that may be present in the product.

The amount of fuel product stream or syngas that is generated by the gasifier and communicated to the fuel cell may vary. For example, from about 1 vol % to about 100 vol % of the fuel product stream from the gasifier may be fed to the fuel cell, or from about 5 vol % to about 75 vol % of the fuel product stream, or from about 15 vol % to about 65 vol % of the fuel product stream, or from about 35 vol % to about 50 vol % of the fuel product stream. The amount of fuel product fed to the fuel cell will depend on the size of the fuel cell and also on degree to which the user may desire to convert the syngas product of the gasifier to carbonaceous fuel. The user of ordinary skill in the art will encounter little difficulty in determining the optimal proportion of the gasifier product stream to divert to the fuel cell, and gasifier product not sent to the fuel cell may be sent to other process units for further combustion or storage.

A variety of carbonaceous materials are suitable for use as feedstocks for the gasifier. Organic materials, such as biomass, are considered particularly suitable. Other carbonaceous materials, including coal, polymers, plastic, rubber, and the like are all suitable. In some embodiments, the feed to the gasifier may include sewage sludge, municipal solid waste, agricultural waste, and the like, including chicken litter, pig manure and cow dung. The feed may comprise a single material or a mixture of materials. The feed may be supplied to the gasifier as a solid or in liquid or slurry form. Coal slurry is considered especially suitable.

The carbonaceous material present in the overall feed may vary. For example, the carbonaceous material may be from 0.01 wt % to about 99 wt % of the overall feed, or from about 10 wt % to about 90 wt %, or even from about 20 wt % to about 80 wt %. A conveyor, pipe, or any other suitable mechanism may be used to introduce the fuel to the gasifer.

In some embodiments, the feed to the gasifier is thermally pretreated, chemically pretreated, or both, to render the feed more suitable for gasification. The pH of the feedstock may be adjusted such that processing of the feed does not adversely impact the process equipment.

Gasification is suitably performed in the range of from about 800 deg. C. to about 1600 deg. C. Lower gasification temperatures may be employed in certain types of gasifiers such as indirectly heated fluidized bed systems; higher temperatures are often seen in devices such as partial oxidation entrained flow gasifiers. Entrained gasifiers can suitably operate at least partially by indirect heating. To perform the Boudouard reaction, CO₂+C=2 CO, temperatures of 800 deg. C. or greater are typically employed. Gasification processes are suitably performed at about 800 deg. C or greater.

Gasification is performed at a wide range of pressures, from e.g., about 1 atm to about 72 atm, or from about 5 atm to about 50 atm, or from about 10 atm to about 25 atm, or even at about 15 atm. The optimal process pressure will be apparent to those ordinary skill in the art and will depend on the desired composition of the product stream from the gasifier and desired throughput.

In some embodiments, water is added to the gasifier during the gasification process. Steam may also be added to the gasifier, suitably such that the molar ratio of steam to carbon present in the material introduced to the gasifier is in the range of from about 1:10 to about 5:1, or even 1:1. A ratio of 1:10 is useful, for example, in a partial oxidation gasifier where a product gas comprising CO is desired; a ratio of 1:1 may be useful where coal/slurry is being gasified. In other embodiments, partial oxidation with little to no water or steam is performed to produce CO and hydrogen, which products evolve from dissociation of the hydrocarbons in the feedstock. While some forms of plasma gasification achieve this, it is nevertheless expected that introducing carbon dioxide into the reaction will enhance the resulting product gas evolution.

A variety of gasification methods are considered suitable. Gasification may be accomplished by, for example, partial oxidation, by steam gasification, by gasification where solar energy provides indirect heating of reactants, feed, and process modules, by plasma-assisted gasification where added CO₂ assists in the gasification. Combinations of gasification methods are also considered suitable.

Various mechanical and hydrodynamic-based configurations of gasifiers are considered suitable. Downdraft and updraft gasifiers are considered suitable, as are fixed bed, moving bed, fluidized bed, bubbling bed, circulating fluidized bed, fast fluidized bed (including a transport reactor having enhanced circulation), entrained flow (both slurry and dry feed), and gasifiers involving partial oxidation or indirect heating are all considered suitable.

Carbon dioxide is controllably added to the gasifier so as to tune the output of the gasifier processing. The carbon dioxide conveyed to the gasifier can be in liquid form. For example, liquid carbon dioxide can be added carbonaceous feed material. The disclosed methods also, in some embodiments, include combining the carbon dioxide of the syngas product stream conveyed to the gasifier with at least a portion of the feed stream prior to introduction to the gasifier. In such embodiments, the liquid carbon dioxide may be used to convey feed material into the gasifier, although such conveyance can also be accomplished with gaseous carbon dioxide. As shown in FIG. 3, the carbon dioxide fed to the gasifier is, at least in part, a product of a fuel cell.

Gasification is suitably performed in the presence of oxygen. The oxygen present in the gasifier may be the oxygen that is present under ambient conditions, or may added, supplemental oxygen. Depending on the user's needs, the gasification may also be performed with or without added oxygen. Where oxygen is used in the reaction, the source of the oxygen may be from any suitable source for the particular application including, for example, ambient air, an oxygen tank, and/or an oxygen generator.

As described elsewhere herein, the syngas product stream of the gasifier includes carbon dioxide. In an exemplary embodiment, at least a portion of the carbon dioxide of the syngas product stream may be conveyed to the gasifier such that carbon dioxide constitutes between about 10 vol % and about 50 vol %, or between about 15 and about 45 vol %, or even between about 20 and about 30 vol % of all material introduced to the gasifier.

In some embodiments, carbon dioxide constitutes between about 20 vol % and 30 vol % of all material introduced to the gasifier. The balance between the carbon dioxide introduced to the gasifier from the first product stream, the syngas product stream, the recycled combusted product from the fuel cell, or all of the above, will depend on the user's needs.

In some embodiments, material is introduced continuously to the gasifier. In other embodiments, material is introduced in a batch or semi-continuous process. Hydrogen, oxygen, or both may be introduced to or removed from the gasifier or other process modules.

Some of the heat contained within the fuel product stream from the gasifier, the product stream from the fuel cell, or both, can be extracted. The fuel cell product stream is a useful source of heat; fuel cell products may be present at up to about 500 deg. C., 1000 deg. C., or greater. Heat may be extracted using any suitable method or mechanism including, for example, by use of a heat exchanger or heat sink. At least a portion of the extracted heat can be supplied to the gasifier, which has the benefit of reducing the amount of other energy that must be expended to heat the gasifier. Transferring heat from one process module to another thus reduces the disclosed methods' need for externally-supplied utilities. The extracted heat can also be used to provide heat to the fuel cell—SOFC units typically operate at comparatively high temperatures—or to other related process units. The fuel cell may be in physical contact with the gasifier so as to ease heat transfer between the two process units. Extracted heat may also be used to heat buildings, residences, or other structures. By extracting and re-using heat taken from the fuel cell, the utility requirements of the overall generation facility can be greatly reduced.

The methods include further processing of the syngas product stream, which further processing, in some embodiments, may include subjecting the syngas product stream to a Fischer-Tropsch reaction scheme. As described elsewhere herein as well as in U.S. provisional application 61/117,988, this reaction scheme enables the production of hydrocarbons from carbon monoxide. In some embodiments, the further processing includes separating carbon dioxide from the syngas product stream, sequestering carbon dioxide from the syngas product stream, or both. The further processing, in certain embodiments, can include combustion. Still further, the further processing may comprise processing at a fuel cell as depicted in FIG. 3.

In exemplary embodiments, controllers are used to manage the flow into and out of the various system components. For example, a controller may be used to modulate the amount of carbon dioxide product that enters the fuel cell. A controller may also be used to modulate the relative proportions of carbon dioxide, oxygen, and feed that enter the gasifier. In some embodiments, a controller or set of controllers can modulate the relative amounts of the gasifier feeds and the operating conditions of the gasifier, fuel cell, and condensers so as to optimize the amount of energy evolved from the fuel cell as compared to the amount of carbon dioxide released, the amount of feed consumed, or other process variables.

Those skilled in the art will appreciate that conduits and/or other suitable mechanisms may be used to communicate reactants between the various system components. For example, at least one conduit may place at least a portion of the carbon dioxide emitted from the combustion chamber in fluid communication with the least one inlet of the gasifier and a controller capable of modulating the carbon dioxide entering the gasifier such that the carbon dioxide entering the gasifier represents between about 10 vol % and 50 vol % of all material entering the gasifier.

Thus, Applicants have disclosed systems and methods that generate electrical power from processing of carbonaceous materials, and cycle carbon dioxide that is created during the generation of electrical power to the processing of carbonaceous materials. A gasifier may be fluidly coupled to a fuel cell. Within the gasifier, at least a portion of a carbonaceous material is at least partially gasified in the presence of carbon dioxide so as to give rise to a fuel product, which may comprise, for example, carbon monoxide and hydrogen. The fuel product is communicated to the fuel cell where at least a portion of the fuel product is employed to generate at least electrical energy and a second product stream comprising carbon dioxide and water. At least a portion of the carbon dioxide evolved from the fuel cell is communicated back to the gasifier where it is used in the processing of carbonaceous material.

The potential embodiments are not limited to the specific devices, methods, applications, conditions or parameters described and/or shown herein, and that the terminology used herein is for the purpose of describing particular embodiments by way of example only and is not intended to be limiting. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Certain features of the potential embodiments which are, for clarity, described herein in the context of separate embodiments, may also be provided in combination in a single embodiment. Conversely, various features of the potential embodiments that are, for brevity, described in the context of a single embodiment, may also be provided separately or in any subcombination. Further, reference to values stated in ranges include each and every value within that range.

What is claimed:

1. A method of processing a carbonaceous material, comprising:
introducing into a gasifier a feed stream comprising a carbonaceous material;
at least partially gasifying at least a portion of the carbonaceous material to give rise to a syngas product stream comprising at least carbon monoxide;
further processing the syngas product stream to give rise to a second product stream comprising at least carbon dioxide; and
conveying at least a portion of the carbon dioxide of the second product stream to the gasifier such that carbon dioxide constitutes between about 10 vol % and about 50 vol % of all the material introduced to the gasifier.

2. The method of claim 1, wherein the carbonaceous material comprises an organic material.

3. The method of claim 1, wherein the feed stream comprises coal.

4. The method of claim 1, wherein the feed stream comprises a polymer, a plastic, a rubber, or any combination thereof.

5. The method of claim 1, wherein the feed stream comprises sewage sludge, municipal solid waste, agricultural waste, or any combination thereof.

6. The method of claim 1, wherein the gasification is performed in the range of from about 800 deg. C. to about 1600 deg. C.

7. The method of claim 1, wherein the gasification is performed at from about 1 atm to about 72 atm.

8. The method of claim 1, further comprising adding water to the gasifier.

9. The method of claim 1, further comprising adding steam to the gasifier.

10. The method of claim 8, wherein the molar ratio of steam to carbon present in the material introduced to the gasifier is in the range of from about 1:10 to about 5:1.

11. The method of claim 1, wherein the gasification is accomplished by partial oxidation, steam gasification, gasification wherein indirect heating is provided by solar heating, plasma assisted gasification, or by any combination thereof.

12. The method of claim 1, wherein conveying at least a portion of the second product stream comprises separating carbon dioxide from the second product stream and transporting at least a portion of said carbon dioxide to the gasifier.

13. The method of claim 12, further comprising sequestering at least a portion of the carbon dioxide of the second product stream.

14. The method of claim 1, wherein at least a portion of the carbon dioxide of the second product stream conveyed to the gasifier enters the gasifier in liquid form.

15. The method of claim 1, further comprising combining the carbon dioxide of the second product stream conveyed to the gasifier with the feed stream prior to introduction to the gasifier.

16. The method of claim 1, wherein landfill gas is used to generate carbon dioxide through steam reformation, combustion, or processing with a solid oxide fuel cell, at least a portion of which carbon dioxide is introduced to the gasifier.

17. The method of claim 1, wherein the gasification is performed in the presence of added oxygen.

18. The method of claim 1, wherein the gasificiation is performed substantially without added oxygen.

19. The method of claim 1, further comprising transporting at least a portion of the carbon dioxide of the syngas product stream to the gasifier such that carbon dioxide constitutes between about 10 vol % and 50 vol % of all material introduced to the gasifier.

20. The method of claim 1, wherein the feed stream is introduced continuously to the gasifier.

21. The method of claim 1, further comprising conveying at least a portion of the carbon dioxide of the second product stream to the gasifier such that carbon dioxide constitutes between about 20 vol % and about 30 vol % of all material introduced to the gasifier.

22. The method of claim 1, wherein the gasification is accomplished by a fluidized bed.

23. The method of claim 1, further comprising addition of hydrogen, oxygen, or both to the gasifier.

24. The method of claim 1, further comprising removal of hydrogen, oxygen, or both from the gasifier.

25. The method of claim 1, further comprising transferring to the gasifier heat evolved from further processing the first product stream.

26. The method of claim 1, wherein the gasification is performed by partial oxidation.

27. The method of claim 1, wherein the further processing comprises deriving a hydrocarbon from the syngas product stream.

28. The method of claim 27, wherein the further processing comprises subjecting the syngas product stream to a Fischer-Tropsch reaction scheme.

29. The method of claim 1, wherein the further processing comprises separating carbon dioxide from the syngas product stream.

30. The method of claim 1, wherein the further processing comprises sequestering carbon dioxide from the syngas product stream.

31. The method of claim 1, further comprising sequestering carbon dioxide from the second product stream.

32. The method of claim 1, wherein the further processing comprises combustion.

33. The method of claim 1, wherein the further processing provides ammonia, a plastic, a polymer, a cellulosic polymer, a solvent, a resin, naphtha, a wax, an alcohol, ethylene, acetate, propylene, a hydrocarbon, or any combination thereof.

34. A system for producing a hydrocarbon from a carbonaceous material, comprising:
a gasifier having at least one inlet and one outlet, the outlet of the gasifier being in fluid communication with a combustion chamber,
the combustion chamber emitting a product stream comprising at least carbon dioxide;
a conduit placing at least a portion of the carbon dioxide emitted from the combustion chamber in fluid communication with the least one inlet of the gasifier,
a controller capable of modulating the carbon dioxide entering the gasifier such that the carbon dioxide entering the gasifier represents between about 10 vol % and 50 vol % of all material entering the gasifier.

35. The system of claim 34, wherein the gasifier is an indirect or a partial combustion gasifier.

36. The system of claim 34, further comprising a continuous supply of feed material to the gasifier.

37. The system of claim 34, further comprising a steam reformer, a combustor, a fuel cell, or any combination thereof, in fluid communication with the gasifier.

38. A system for producing power, comprising:
a gasifier having at least one inlet and one outlet, the outlet of the gasifier being in fluid communication with a fuel cell,
the fuel cell capable of emitting a product stream comprising at least carbon dioxide;
a conduit placing at least a portion of the carbon dioxide emitted from the fuel cell in fluid communication with the least one inlet of the gasifier,
a controller capable of modulating the carbon dioxide entering the gasifier such that the carbon dioxide entering the gasifier represents between about 1 vol % and 100 vol % of all material entering the gasifier.

39. The system of claim 38, wherein the fuel cell comprises a solid oxide fuel cell, a molten carbonate fuel cell, a protonic ceramic fuel cell, or any combination thereof

40. The system of claim 39, wherein the fuel cell comprises a solid oxide fuel cell.

41. The system of claim 38, further comprising placement of the fuel cell product stream and the gasifier in thermal communication with one another.

42. The system of claim 38, further comprising a condenser in fluid communication with the fuel cell.

43. The system of claim 38, wherein the gasifier and fuel cell are in physical contact with one another.

44. The system of claim 38, further comprising a heat exchanger capable of transferring heat between the gasifier and fuel cell.

45. A method of producing electrical energy, comprising:
in a gasifier, at least partially gasifying at least a portion of a carbonaceous material in the presence of carbon dioxide so as to give rise to a fuel product comprising carbon monoxide and hydrogen;
operating a fuel cell so as to convert at least a portion of the fuel product to at least electrical energy and a second product stream comprising carbon dioxide and water; and
recirculating at least a portion of the carbon dioxide evolved from the fuel cell to the gasifier.

46. The method of claim 45, wherein from about 1 vol % to about 100 vol % of the carbon dioxide evolved from the fuel cell is recirculated to the gasifier.

47. The method of claim 45, wherein from about 10 vol % to about 80 vol % of the carbon dioxide evolved from the fuel cell is recirculated to the gasifier.

48. The method of claim 45, further comprising isolating at least a portion of the carbon dioxide present in the second product stream.

49. The method of claim 45, wherein the isolating is accomplished by condensing at least a portion of the second product stream.

50. The method of claim 45, wherein from about 1 vol % to about 100 vol % of the fuel product stream is fed to the fuel cell.

51. The method of claim 50, wherein from about 5 vol % to about 75 vol % of the fuel product stream is fed to the fuel cell.

52. The method of claim 45, wherein the carbonaceous material comprises coal, biomass, or any combination thereof.

53. The method of claim 45, further comprising extracting at least a portion of heat contained within the fuel product stream, the second product stream, or any combination thereof.

54. The method of claim 53, further comprising supplying at least a portion of the extracted heat to the gasifer.

55. A method of producing a hydrocarbon from a carbonaceous material, comprising:
introducing a volume of carbonaceous material into a gasifier;
at least partially gasifying at least a portion of that material so as to evolve a first product stream comprising at least carbon monoxide;
processing the carbon monoxide of the product stream with hydrogen to give rise to a second product stream comprising at least carbon dioxide and a hydrocarbon; and
introducing carbon dioxide into the gasifier such that carbon dioxide constitutes between about 10 vol % and about 50 vol % of all material introduced to the gasifier.

56. The method of claim 55, wherein the introducing the carbon dioxide into the gasifier is performed such that carbon dioxide constitutes between about 20 vol % to about 30 vol % of all material introduced to the gasifier.

57. The method of claim 55, wherein waste gas is used to generate carbon dioxide through steam reforming of the waste gas, combustion of the waste gas, or processing the waste gas with a solid oxide fuel cell, at least a portion of which carbon dioxide is introduced to the gasifier.

58. The method of claim 57, wherein the waste gas comprises landfill gas.
